(19) United States
(12) Patent Application Publication
(10) Pub. No.: US 2015/0126821 A1
(43) Pub. Date: May 7, 2015

(54) Support System And Method For Detecting Neurodegenerative Disorder

(71) Applicant: Technical University of Denmark, Kgs. Lyngby (DK)

(72) Inventors: Lykke Kempfner, Herlev (DK); Poul Jørgen Jennum, Farum (DK); Helge Bjarup Dissing Sørensen, Graested (DK)

(21) Appl. No.: 14/407,340

(22) PCT Filed: Jun. 12, 2013
(86) PCT No.: PCT/EP2013/062164

§ 371 Date: Dec. 11, 2014

(30) Foreign Application Priority Data
Jun. 12, 2012 (EP) 12171637.7

(51) Int. Cl. A61B 5/00

(52) U.S. Cl. CPC A61B 5/4812; A61B 5/4082; A61B 5/4088
USPC 600/301

(57) ABSTRACT

The present invention relates to a system and a method for detection of abnormal motor activity during REM sleep, and further to systems and method for assisting in detecting neurodegenerative disorders such as Parkinson's. One embodiment relates to a method for detection of abnormal motor activity during REM sleep comprising the steps of: performing polysomnographic recordings of a sleeping subject, thereby obtaining one or more electromyography (EMG) derivations, preferably surface EMG recordings, and one or more EEG derivations, and/or one or more electrooculargraphy (EOG) derivations, detecting one or more REM sleep stages, preferably based on the one or more EEG and/or EOG derivations, determining the level of muscle activity during the one or more REM sleep stages based on the one or more EMG derivations, wherein a subject having an increased level of muscle activity during REM sleep compared to one or more normal subjects has abnormal motor activity during REM sleep.

The present invention relates to a system and a method for detection of abnormal motor activity during REM sleep, and further to systems and method for assisting in detecting neurodegenerative disorders such as Parkinson's.

### BACKGROUND OF INVENTION

Synucleinopathies are neurodegenerative disorders characterized by Lewy bodies and include Parkinson's disease, dementia with Lewy bodies and multiple system atrophy.

Parkinson's disease (PD) is a degenerative disorder of the central nervous system. The prevalence of PD is approximately 0.5% to 1% among people 65 to 69 years of age, rising to 1% to 3% among those aged 80 years or older. The neurodegeneration occurring in PD is irreversible and there is currently no cure for the disease.

The most obvious symptoms of PD are movement-related and include unilateral tremor, rigidity, akinesia and postural instability. Later, cognitive and behavioural problems may arise, with dementia commonly occurring in the advanced stages of the disease. Other symptoms include sensory, sleep and emotional problems.

Diagnosis of PD is currently based on the clinical manifestation of the motor symptoms, and treatments are directed at managing clinical symptoms. When the diagnosis is made based on the manifestation of the motor symptoms, the brain is already severely affected as the motor symptoms of PD arise from the loss of dopamine-generating neurons in the substantia nigra.

There are currently no reliable screening techniques available, which are capable of detecting PD in its very early stages, i.e. before motor symptoms appear. Such early screening techniques could potentially lead to the identification of more efficient treatments of Parkinson's disease and possible to a cure.

Rapid eye movement Sleep Behavior Disorder (RBD) is characterized by REM sleep without atonia (RSWA) and/or dream enactment. Therefore increased muscle tone and excessive phasic muscle-twitch activity of the submental or limb surface electromyograph (EMG) may be measured.

RBD without any current sign of neurodegenerative disorder is designated as idiopathic RBD (iRBD). This term is questionable since RBD and other non-motor symptoms and findings are often observed in Parkinson's disease (PD) and atypical PD, such as multiple system atrophy (MSA) and Lewy Body Dementias (LBDs). Additionally, more than 50% of the subjects diagnosed with iRBD will develop a synucleinopathy within 5-10 years. Correct detection of RBD is therefore highly important, provided that neuroprotective treatment becomes available.

Rapid eye movement sleep behaviour disorder (RBD) affects about 0.4% of adults, 0.5% of older adults, 33% of patients with newly diagnosed Parkinson's disease, and 90% of patients with multiple system atrophy. Consequences can include injury to the patient, threats to the safety of a bed partner, and inability to share a bed with a partner. Diagnosis is important because the condition responds well to treatment, most often with clonazepam. Moreover, RBD may be a harbinger for neurodegenerative conditions such as Parkinson's disease (PD), multiple system atrophy (MSA), or dementia with Lewy bodies (DLB), which together comprise the alpha-synucleinopathies. In the absence of RBD, REM sleep without atonia may also signal increased risk for alpha-synucleinopathies.

REM behaviour Disorder, dream enacting behaviour and abnormal muscle activity during REM sleep, may be early markers for neurodegenerative diseases, such as Parkinson's disease and atypical PD. More than 50% of the subjects diagnosed with RBD will develop PD within a time span of 5-10 years.

Hence, an improved support system for allowing health care persons to provide a diagnosis to patients as early as possible would be advantageous, and in particular a more efficient and/or reliable method for this would be advantageous.

### SUMMARY OF INVENTION

There is a need for identification of novel biomarkers for synucleinopathies allowing for an earlier detection of these diseases. Such early detection could potentially lead to the development of novel and more efficient treatments and eventually to a cure.

A first aspect of the invention therefore relates to a method for detection of abnormal motor activity during REM sleep comprising the steps of:
performing polysomnographic recordings of a sleeping subject, thereby obtaining one or more electromyography (EMG) derivations, preferably surface EMG recordings, and one or more EEG derivations, and/or one or more electrooculargraphy (EOG) derivations,
detecting one or more REM sleep stages, preferably based on the one or more EEG and/or EOG derivations,
determining the level of muscle activity during the one or more REM sleep stages based on the one or more EMG derivations,
wherein a subject having an increased level of muscle activity during REM sleep compared to one or more normal subjects has abnormal motor activity during REM sleep.

A further embodiment of the invention relates to a system for detection of abnormal motor activity during REM sleep comprising sets of EMG and EEG and/or EOG electrodes for recording a dataset of polysomnographic signals of a sleeping subject, a processing unit configured for detecting one or more REM sleep stages, preferably based on at least a part of said dataset of polysomnographic signals, determining the level of muscle activity during the one or more REM sleep stages based on EMG data in said dataset, and determining whether the subject is having an increased level of muscle activity during REM sleep compared to one or more normal subjects.

Abnormal motor activity during REM sleep may be an indicator for synucleinopathy or early stages therefore. A further aspect of the invention therefore relates to a method for identifying a subject having an increased risk of developing a synucleinopathy comprising detecting abnormal motor activity during REM sleep according to the above described system or method, wherein a subject having an abnormal motor activity during REM sleep has an increased risk of developing a synucleinopathy.

A further aspect of the invention relates to a method and system that provides a reliable output allowing a heath care person to assess the risk that a given patient suffers from neurodegenerative decease, e.g. a method for assessing sleep and/or wake patterns in a person, the method comprising:
recording a set of physiological signals in a time interval,
applying a filter to the set of physiological signals so as to reduce artefact and/or noise from the set of physiological signals,
classifying a sleep and/or wake pattern in one or more sub-time intervals of the time interval, wherein the classification is based on a signal from the set of physiological signals and wherein the sleep and/or wake pattern is classified as a first type, such as REM sleep, or a second type, such as NREM sleep,
classifying a motor pattern in each of the one or more sub-time intervals based on a signal from the set of physiological signals and the sleep and/or wake type into a first motor pattern type, such as normal muscle activity during REM sleep or REM sleep with atonia, or a second motor pattern type, such as increased muscle activity during REM sleep or REM sleep without atonia, and
computing a motor descriptor, such as a motor descriptor relating to rapid eye movement Sleep Behavior Disorder, based on the motor pattern type.

Yet a further embodiment of the invention relates to a system for assisting a health care person in assessing sleep and/or wake patterns in a person, the system comprising a set of electrodes for recording a set of physiological signals in a time interval, a filter for filtering the set of physiological signals so as to reduce artefact and/or noise from the set of physiological signals, a pattern classifier device for classifying a sleep and/or wake pattern in one or more sub-time intervals of the time interval, wherein the classification is based on a signal from the set of physiological signals and wherein the sleep and/or wake pattern is classified as a first type or a second type, a motor classifier for classifying a motor pattern in each of the one or more sub-time intervals based on a signal from the set of physiological signals and the sleep and/or wake type into a first motor pattern type or a second motor pattern type, and a processor for computing a motor descriptor based on the motor pattern type.

In one embodiment, the present invention relates to a method for identifying a subject having an increased risk of developing a synucleinopathy, such as Parkinson's disease, preferably based on said computed motor descriptor.

### DETAILED DESCRIPTION OF THE INVENTION

Early detection of PD may thus be provided by considering abnormally high muscle activity during REM sleep. In one embodiment of the invention the detection is provided by means of outlier detection implicitly assuming that surface EMG (sEMG) activity is absent during REM sleep, and muscle activity can be regarded as being outliers. Quantifying these outliers during REM sleep may be an efficient method to identify RSWA. In one embodiment of the invention the applied electrodes are the submentalis (CHIN), the left and right EOG (EOGL, EOGR), and the left and right anterior tibialis (TIBL, TIBR) totaling five sEMG electrodes. The two EOG channels are normally used for monitoring eye movements, such as REMs during REM sleep, but in here they may be reused for measuring facial muscle activity.

When detecting RSWA the subjects are usually equipped with EMG electrodes on the legs, because the legs typically move during REM sleep without atonia. However, the legs may move too much for subjects with RSWA possibly causing the electrodes to fall off. As demonstrated in example 1 herein RSWA may be detected by using EMG signals from CHIN and/or EOG electrodes only. Thus, the level of muscle activity determined during REM sleep may be based on eye movements only, and/or submentalis movements only and/or eye movements and submentalis movements only. By skipping the TIB electrodes the measurement procedure becomes much more cost efficient and simpler, thus more patients can be diagnosed for the same costs. Thus, in one embodiment of the invention said one or more electromyography (EMG) derivations are derived from a CHIN EMG electrode only, from eye movements only and/or from eye movement and a CHIN EMG electrode only. The eye movements may be recorded by means of EOG electrodes, preferably an EOG-L and an EOG-R electrode. I.e. extracting EMG signals from EOG electrodes.

The RSWA detection can be automated if the motor activity during REM sleep can be classified as e.g. a REM sleep with atonia (normal REM sleep) or REM sleep without atonia (RSWA). E.g. fully automated, i.e. it does not involve manual analysis of the EEG derivations by a sleep expert. Thus, in a further embodiment a motor activity is classified, such as a muscle activity, in a plurality of time intervals (mini epocs) of the REM sleep stages, based on one or more of the EMG derivations, into a first motor activity type, such a REM sleep with atonia, or a second motor activity type, such as REM sleep without atonia (RSWA). Thus, the increased level of muscle activity during REM sleep may be detected based on the classification of the time intervals during REM sleep stages into said first and second motor activity types, e.g. an increased level of muscle activity during REM sleep may be detected based on the number of time intervals characterized as first and/or second motor activity types.

Thus, for example an increased level of muscle activity during REM sleep may be detected based on the number of time intervals characterized as first motor activity type relative to the number of time intervals characterized as second motor activity type. In a further embodiment of the invention a single motor activity score, such as a single number, is computed for the subject based on the classification of the time intervals during REM sleep into first and second motor activity types and wherein an abnormal motor activity is detected for said subject based on said single score. The motor activity score may be based on the number of time intervals characterized as first motor activity type relative to the number of time intervals characterized as second motor activity type.

The duration of each of said time intervals may be between 1 and 60 seconds, or between 1 and 30 seconds, or between 1 and 10 seconds, such as 1, 2, 4, 5, 6, 7, 8, 9, or 10 seconds, preferably 3 seconds.

The classification may be based on a supervised learning model, such as a support vector machine algorithm, such as the one-class support vector machine (OC-SVM) classifier. The classification may therefore be based on outlier detection, wherein muscle activity during REM sleep is defined as being outlier, or a predefined muscle activity during REM sleep is defined as being outlier, or abnormal muscle activity during REM sleep is defined as being outlier.

The level of muscle activity measured during REM sleep may be compared to the level of muscle activity during REM sleep in a group of healthy subjects or to a previous measurement of the level of muscle activity during REM sleep in the same subject. In a further embodiment of the invention an increased level of muscle activity during REM sleep is increased by about a factor 1.5 or more compared to control, for example about a factor 2 or more, or about a factor 3 or more, or about a factor 4 or more, or about a factor 5 or more.

A filter may be applied to the EMG and EEG and/or EOG signals, so as to reduce artefact and/or noise from the set of physiological signals. The filter may be a band-pass filter, such as a fourth-order Butterworth filter with a cut-off frequency such as 30 Hz and 60 Hz respectively. The filter may be a notch filter, such as a fourth-order Butterworth notch-filter with a cut-off frequency.

As stated above the present invention further relates to a method for identifying a subject having an increased risk of developing a synucleinopathy wherein abnormal motor activity during REM sleep is detected and wherein a subject having an abnormal motor activity during REM sleep has an increased risk of developing a synucleinopathy. The subject may thus be identified before clinical onset of the synucleinopathy. The synucleinopathy is selected from Parkinson's disease, Multiple System Atrophy and Dementia with Lewy Bodies. The subject may thus be identified before manifestation of one or more motor symptoms selected from the group consisting of tremor, rigidity, akinesia and postural instability. The subject is preferably identified before substantial neurodegeneration has occurred.

A person who is to be given a risk assessment by a health care person may be fitted with a number of electrodes so that an appropriate apparatus records a set of physiological signals in a time interval. This time interval may be a short or longer period of time. In some embodiments the period of time may be a full night sleep. As persons suffering from a neurodegenerative disease may have an interrupted sleep pattern there may be employed some automatic method for detecting when the person being monitored is in a specific sleep state.

The sleep stages, e.g. REM sleep, NREM (or non-REM) sleep, or wake, may be detected manually, semi-automatically or fully automatically, e.g. based on hypnograms. A computerized sleep-stage detector may be provided, such as the one described herein below and also proposed by Kempfner et al. in 2012 employing standard EEG and EOG channels.

The signals recorded may be subject to filtering so as to reduce artefacts. The filter may be the same filter applied to all channels or the filtering step may include filtering each signal in the set of physiological signals in a specific way.

The recorded signal may be divided into one or more sub-intervals wherein a sleep and/or wake pattern may be established. Thereby the signals recorded may be classified so that a general overview of the recording may be generated. The stages are classified as at least first type, e.g. REM sleep, or a second type, e.g. NREM sleep. Further types may be established so as to more finely classifying the sub-intervals.

In each of these sub-intervals a motor pattern may be determined for each of the one or more sub-time intervals. This motor pattern provides some information regarding the motor activity of the person in the related time period. Examples of motor patterns are muscle activity patterns, such as specific muscle activity patterns, such as increased level of muscle activity, such as normal level of muscle activity, such as high muscle activity, such as abnormal high muscle activity, such as abnormal muscle activity observed during REM sleep which may be connected with RSWA. A motor pattern may be muscle activity intensity, duration, pattern, etc., from one or more of the following muscles: submentalis, left/right anterior tibialis, and/or from one or more of the muscles controlling eye movement. Also muscles from the arms may be monitored. Muscle activity may be monitored by means of EMG, preferably surface EMG (sEMG).

A motor pattern may be classified into a motor pattern type, e.g. by means of an automatic classifier. Examples of motor pattern types are “REM sleep with atonia” and “REM sleep without atonia”. The absolute or relative number of various motor pattern types may lead to the determination of a motor descriptor, which may provide a single number indicating whether the patient suffers from a particular disorder.

The method determines a motor descriptor from which the health care person is assisted in making a full evaluation of the person and allowing the health care person to establish a risk factor that the person suffers from a neurodegenerative disease, such as a synucleinopathy, such as Parkinson's disease. A motor descriptor may be related to idiopathic rapid eye movement (REM) sleep Behavior Disorder (iRBD), which is a strong early marker of Parkinson's disease and the motor descriptor is preferably based on the classification of the motor patterns into types. The method provides an indication that the risk is high earlier that the methods presently used for determining the risk that a person suffers from a neurodegenerative decease.

In an embodiment the set of physiological signals may be a combination of one or more of the following physiological signals, namely muscle activity at or near the eye, eye movement morphology, muscle activity measured from one or more body parts including limbs and head, respiration frequency, heart rate, an electroencephalographycal (EEG) signal, an electrooculographycal (EOG) signal, an eletrocardiographycal (ECG) signal and/or an electromyographycal (EMG) signal. The above-mentioned signals may be combined when using the method according to the present invention.

In an embodiment the classification of the sleep stage, in one or more sub-time intervals based on a signal from the set of physiological signals, may be based on one or more of the following classification methods: Linear and/or nonlinear classifiers including, but not limited to, neural Network, Support Vector Machine, k-Nearest Neighbour and/or Bayes Classifiers. Further, the method may be combined, e.g. some methods may work better for certain distribution of signals in the various sub-intervals.

Advantageously the physiological signal may be an electrophysiological signal.

If applying at least one filter to the set of physiological signals it is possible to reduce artifacts and/or noise. The noise could e.g. be generated from power line, electrical equipment, or other parts of body such as the heart or muscles.

When filtering the signal multiple filters may be applied. The filters may be applied sequentially or in parallel. In some embodiments the filter may be a band-pass filter. In an embodiment the band-pass filter may be a fourth-order Butterworth filter with a cut-off frequency around 30 Hz and 60 Hz respectively, and in further embodiments the cut-off frequency may be 100 Hz or above. In some embodiments the filter may be a notch filter.

In an embodiment the filter may be a fourth-order Butterworth notch filter with a cut-off frequency such as 48 Hz and 52 Hz respectively. Other filter characteristics may be used. For each of the frequency levels an interval around the suggested values may be used. The interval may have a range of ±10 Hz at the above-mentioned frequencies.

The method according to the first aspect may be implemented in a computer program product being adapted to enable a computer system comprising at least one computer having data storage means in connection therewith to control an apparatus according to the second aspect of the invention. Such a computer program product may be provided on any kind of computer readable medium, or through a network, including on an optical storage medium, a network accessible disk or server.

A further aspect of the present invention relates to a system for assisting a health care person in assessing sleep and/or wake patterns in a person. The system may comprise a set of electrodes for recording a set of physiological signals in a time interval, a filter for filtering the set of physiological signals so as to reduce artefact and/or noise from the set of physiological signals, a pattern classifier device for classifying a sleep and/or wake pattern in one or more sub-time intervals of the time interval, wherein the classification is based on a signal from the set of physiological signals and wherein the sleep and/or wake pattern is classified as a first type, such as REM sleep, or a second type, such as NREM sleep, a motor classifier for classifying a motor pattern in each of the one or more sub-time intervals based on a signal from the set of physiological signals and the sleep and/or wake type into a first motor pattern type, such as normal muscle activity during REM sleep or REM sleep with atonia, or a second motor pattern type, such as increased muscle activity during REM sleep or REM sleep without atonia, and a processor for computing a motor descriptor, such as a motor descriptor relating to rapid eye movement Sleep Behavior Disorder, based on the motor pattern type.

The classifiers and/or the processor may be embodied in a general purpose processor or a specialised signal processor or a specialised processor, such as an FPGA. Other types of suitable processor may be used. The processors and/or classifiers communicate via data connections in the system. This may e.g. be via a data bus or other type of communication device. The system is preferably embodied as a single component but individual functions may be distributed to e.g. specialised equipment. The signals recorded may be processed while recording or the data may be analysed at a point in time after the data has been recorded. The post-processing could be advantageous as it allows the system to better determine or distinguish between certain states in the sleep/wake patterns being monitored.

Advantageously the set of electrodes may be arranged to be positioned so as to obtain a signal characterizing one or more of:
muscle activity at or near the eye,
eye movement morphology,
muscle activity measured from one or more body parts including limbs and head,
respiration frequency,
heart rate,
an electroencephalographycal (EEG) signal,
an electrooculographycal (EOG) signal,
an eletrocardiographycal (ECG) signal and/or
an electromyographycal (EMG) signal.

The set of electrodes may be positioned to monitor and record signals from multiple sites on the body of the person being monitored.

In an embodiment the filter may be a band-pass filter. This could be advantageous as certain signals usually occur in certain frequency bands. In an embodiment the band-pass filter may be a fourth-order Butterworth filter with a cut-off frequency such as 30 Hz and 60 Hz respectively. It has proved advantageous to use a filter having these characteristic values.

The individual aspects of the present invention may each be combined with any of the other aspects. These and other aspects of the invention will be apparent from the following description with reference to the described embodiments.

### DESCRIPTION OF DRAWINGS

The present invention will now be described in more detail with regard to the accompanying figures. The figures show one way of implementing the present invention and is not to be constructed as being limiting to other possible embodiments falling within the scope of the attached claim set.

FIG. 1 is a schematic illustration of steps of a method according to one embodiment of the present invention.

FIG. 2 schematically illustrates a person wearing a set of electrodes. The electrodes are placed as EEG, ECG and EMG electrodes.

FIG. 3 is a schematic illustration of a system for assisting a health care person in assessing sleep and/or wake patterns in a person.

FIG. 4 depicts the six Braak stages of Parkinson's disease and the clinical symptoms associated with the different stages. Currently, Parkinson's disease is diagnosed upon manifestation of motor symptoms.

FIGS. 5-11 relate to example 1:

FIG. 5 is a flowchart of a RSWA detector.

FIG. 6 shows a sliding reference window comprising 11 mini-epochs, while the central test window consists of 1 mini-epoch. The step size is one mini-epoch.

FIG. 7 shows the muscle feature extraction process using the CHIN and EOGR channels from the same REM sleep epoch of an iRBD subject.

FIG. 8 shows an example of scatterplots of given subjects during REM sleep from one healthy control (A), one PLM (B) and one iRBD subject (C).

FIG. 9 illustrates the results of a leave-one-subject-out-cross-validation.

FIG. 10 shows validation muscle scores during REM sleep of one random selected run (36 subjects).

FIG. 11 shows test muscle scores during REM sleep (12 subjects).

### DETAILED DESCRIPTION OF THE DRAWINGS

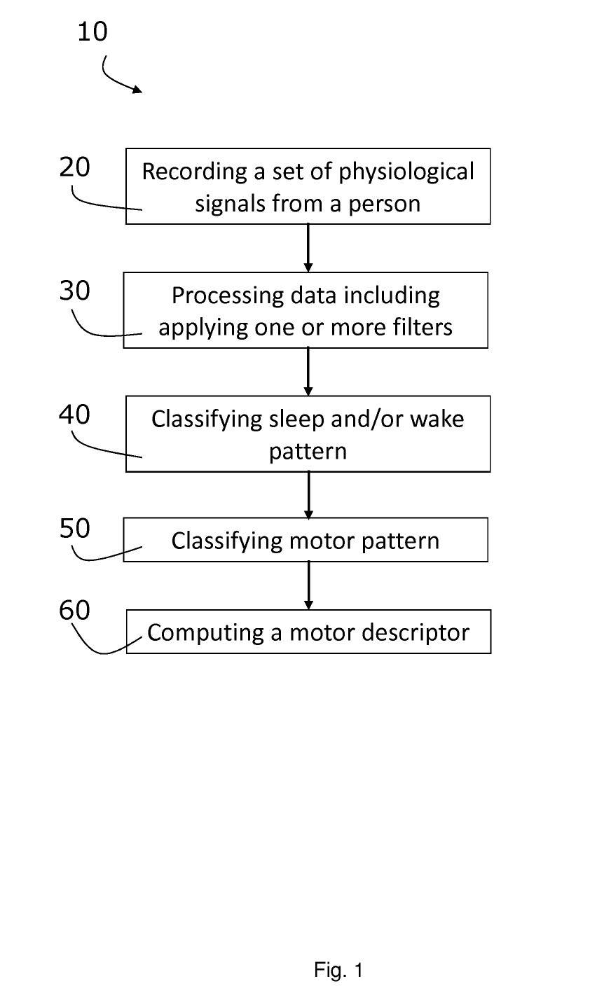

FIG. 1 schematically illustrates steps of a method 10 according to the present invention. The method 10 is especially suitable for assessing sleep and/or wake patterns in a person. The method 10 comprises the step of recording 12 a set of physiological signals in a time interval. This may e.g. be done using a set of electrodes positioned on the person for whom the health care person is to assess sleep and/or wake patterns. The method 10 comprises the step of applying 14 a filter to the set of physiological signals so as to reduce artefact and/or noise from the set of physiological signals. This allows better processing of the signals. The method 10 comprises the step of classifying 16 a sleep and/or wake pattern in one or more sub-time intervals of the time interval, wherein the classification is based on a signal from the set of physiological signals and wherein the sleep and/or wake pattern is classified as a first type or a second type. As the set of signals may be recorded during a long period, and the person being monitored is usually not in the same state throughout the period, it is advantageous to break the measurement down into a number of smaller periods. This also allows the period to be broken down to periods where the stage may be classified as being a first or second type. The first and the second type are different.

The method 10 comprises the step of classifying 18 a motor pattern in each of the one or more sub-time intervals based on a signal from the set of physiological signals and the sleep and/or wake type into a first motor pattern type or a second motor pattern type. The motor pattern provides some information regarding the person's movement in that particular period of time. The method 10 comprises the step of computing 18 a motor descriptor based on the motor pattern type. This motor descriptor is then presented to the health care person so that he is provided with further information that allows him or her to make a full assessment of the person's likelihood, or risk, that the person is in an early stage of a neurodegenerative disease.

In an embodiment of the present invention the set of physiological signals recorded are EEG-signals, ECG-signals, and/or EMG-signals, and the respiratory frequency. The recordings could be performed as a full night polysomnography (PSG) in accordance to the AASM-standard. The EEG is measured from the F3-A2, C3-A2 and O1-A2 EEG channel, where A2 denotes the right mastoid. The left and right EOG channel is used for measuring muscle activity. Standard ECG and respiratory recordings are also performed. The sampling frequency for the recordings is 256 Hz.

The electrodes are connected to a recording device which preferably is a portable device which enables the patient to have the electrodes attach at a clinic and still take the device home for overnight recordings in the home. Some persons are not comfortable sleeping in a non-familiar setting.

The method 10 may comprise automatic detection or determination of when the patient is in REM sleep state. Detect REM sleep stage may be accomplished according to the new international sleep-scoring standard from the American Academy of Sleep Medicine (AASM).

In one embodiment of the present invention the filter is applied in order to reduce the noise generated from the power-line. Therefore a 4th order Butterworth bandpass filter is applied, using a 3 dB zero-phase filtering approach. The chosen cut-off frequencies are 48 Hz and 52 Hz respectively.

The left and right EOG channel denoted EOGL and EOGR respectively are band-pass filtered by the use of 4th order Butterworth bandpass filters using the zero-phase filtering approach. The frequency bands and the denotation are defined in table 1 below.

TABLE 1

| Modality | Band | Low-Cut (3 dB) | High-Cut (3 dB) |
|---|---|---|---|
| EOGL | ρ^{L} | 1 Hz | 10 Hz |
| EOGL | φ^{L} | 10 Hz | 45 Hz |
| EOGR | ρ^{R} | 1 Hz | 10 Hz |
| EOGR | φ^{R} | 10 Hz | 45 Hz |

The ρ-band (1-10 Hz) is assumed to characterize REM, while the φ-band (10-45 Hz) is assumed to contain EMG activity. The EMG signals are viewed in the frequency band 10-100 Hz.

During REM sleep the EEG has low amplitude with mixed frequency content, with a frequency range of approximately 4-7 Hz. However, this may also occur in the Non-REM (NREM) sleep. The EEG channels (F₃-A₂, C₃-A₂, O₁-A₂) are filtered by the use of 4^{th} order Butterworth bandpass filters, using the zero-phase filtering approach. The frequency bands are defined in table 2 below

TABLE 2
Frequency bands of the EEG channels

| Modality | Band | Low-Cut (3 dB) | High-Cut (3 dB) |
|---|---|---|---|
| EEG | δ | 1 Hz | 4 Hz |
| EEG | θ | 4 Hz | 8 Hz |
| EEG | α | 8 Hz | 13 Hz |
| EEG | β | 13 Hz | 30 Hz |
| EEG | γ | 30 Hz | 45 Hz |

The surface detected signals are often affected by noise from slow movement and instability of the electrode-skin interface which typically can be seen in the range of 0-20 Hz. Furthermore many signals are also affected by the power line noise (i.e. 50 Hz in Europe). To reduce the interferences all the signals are preprocessed using a band-pass filter and a notch filter. The applied band-pass filter is in one embodiment of the invention an 8^{th} order Butterworth filter with a 3 dB cut off frequency of 20 Hz and 60 Hz respectively, and the notch filter is a 2^{nd} order Butterworth notch filter with a 3 dB cut off frequency of 48 Hz and 52 Hz respectively.

In REM sleep the eyes tend to move rapidly sideways under closed eyelids. This produces rapid conjugated eye movements, which appear as “out-of-phase” EOG channel deflections. As described above, the EOG channels measures the eye movements, but they also measure the muscle activity (i.e. EMG). This superposition property may be exploited to characterize the increased muscle activity during wake

A 4th order Butterworth band-pass filter with cutoff frequencies at 1 and 5 Hz respectively is used to separate the REM from Slow-Eye-Movement (SEM), baseline drift and EMG activity.

Classifying a sleep and/or wake pattern in one or more sub-time intervals of the time interval, wherein the classification is based on a signal from the set of physiological signals and wherein the sleep and/or wake pattern is classified as a first type or a second type,

Sleep stage is detected according to the international sleep-scoring standard from the American Academy of Sleep Medicine (AASM).

The muscle tone is analyzed by computing the inter-quartile-range (IQR) of the φ^{L} and φ^{R} band. The IQR is less affected by noise and artifacts, such as e.g. EMG and ECG artifacts. That is, if the difference between the 3^{rd} quartile (75^{th} percentile) and 1^{st} quartile (25^{th} percentile), for vector X is defined by:

Q₃₁≡Q₃(X)−Q₁(X) (1)

then the IQR features of φ^{L} and φ^{R}, which are more likely to represent the muscle tone, is given by:

Yₙ^{φ}^{^{L}}=Q₃₁(φₙ^{L}) (2)

Yₙ^{φ}^{^{R}}=Q₃₁(φₙ^{R}) (3)

where 1≦n≦N denotes the mini-epoch number of total N mini-epochs.

The characterization of the rapid-eye-movements is analyzed by computing the normalized-cross-correlation-coefficients of lag 0 (NCCC) of the ρ^{L} and ρ^{R} band respectively. The NCCC is given by:

$${C}_{n} = \frac{cov ( {ρ}_{n}^{L} , {ρ}_{n}^{R} )}{\sqrt{var ( {ρ}_{n}^{L} ) var ( {ρ}_{n}^{R} )}} \qquad ( 4 )$$

where coy denotes the sample covariance between the two variables, while var denotes the sample variance.

The three EEG channels are analyzed with respect to the clinical bands defined in Table (IV) using the IQR approach defined in (1):

Yₙ^{δ}=Q₃₁(δₙ) (5)

Yₙ^{θ}=Q₃₁(θₙ) (6)

Yₙ^{α}=Q₃₁(αₙ) (7)

Yₙ^{β}=Q₃₁(βₙ) (8)

Yₙ^{γ}=Q₃₁(γₙ) (9)

A total of 3 EOG features are computed and 5 EEG features from 3 EEG channels are computed. The features are then merged into a [1×18] feature vector denoted Fₙ^{S}, where S={1, 2, . . . , 16}S={1, 2, . . . , 16} denotes the subject number.

To avoid features with greater dynamic range dominating those with smaller dynamic range, each feature was re-scaled into the range of approximately [0,1]. That is, the individual features from each subject were re-scaled according to a modified min-max method. The subject-specific feature scaling is given by:

$${\tilde{F}}^{S} = \frac{{F}^{S} - min ( {F}^{S} )}{max ( {F}^{S} ) - min ( {F}^{S} )} \qquad ( 10 )$$

Since the original min-max scaling method is not robust towards outliers, the minimum and maximum were estimated by computing the quartiles of the individual features. The upper and lower boundary is defined by:

min(F^{S})defmin(F^{S})≡Q₁(F^{S})Q₁(F^{S}) (11)

max(F^{S})≡Q₃(F^{S}) (12)

Again, several good percentile combinations have been tested, but the 25^{th} percentile and 75^{th} percentile did perform best on our data.

The objective of a classifier is to classify the feature samples into the respective classes, i.e. the REM sleep versus every-thing else. The standard Nearest Neighbor classifier (k-NN) identify the k nearest points from the training data set and then assign a new test point to the class having the largest number of representatives among this set. In this study the Euclidian distance was used to measure the distance between points [18]. The k-NN is a widely used supervised learning method and has successfully been applied to different areas, including sleep analysis [19].

The manual hypnogram was modified into a target vector by first labeling the REM sleep epochs ‘+2’ and everything else ‘+1’. The target vector was then extended by successfully repeating each epoch 10 times. This increase the “sampling rate” from one scoring per 30 second (epoch) to 10 scorings per 30 second, which is equivalent to 1 scoring per 3 second (mini-epoch), cf. FIG. 1). In this study the k-NN classifier was used and the k variable was found by using a simple grid-search approach combined with the leave-one-out cross validation scheme. Since the samples from each subject may be correlated, a fold consists of whole subjects. A single fold was held out for testing, while the remaining 15 folds were used for training. This is done 16 times, so each fold is used for testing. The individual subject detections were then post-processed.

The agreement between the detected REM sleep and the manually scored REM sleep (i.e. hypnogram) was improved by post-processing the k-NN classifier detection. Normally, the NREM and REM sleep tend to alternate through the night in cyclial fashion, where REM sleep usually occur in 4-6 discrete episodes each lasting between 5-20 min [20]. This trend was enhanced by post processing the REM sleep candidates from the detection y of the classifier. Two methods were analyzed with respect to no post-processing; the moving average and the voting method. The moving average method was obtained by filtering the binary detection of each individual subject with a normalized Blackman window defined as:

$$w ( m ) = \frac{1}{τ} ( 0.42 - 0.5 cos ( \frac{2 π m}{M} ) + 0.08 cos ( \frac{4 π m}{M} ) ) \qquad ( 13 )$$

Where 1≦m≦M and M is the estimated duration (odd number) of the Blackman window. The τ corresponds to the normalization coefficient defined as:

$$τ = {\sum }_{m = 0}^{M} ( 0.42 - 0.5 cos ( \frac{2 π m}{M} ) + 0.08 cos ( \frac{4 π m}{M} ) ) \qquad ( 14 )$$

Then the post-processed detection is defined by the convolution:

\tilde{y}(n)≡w*y (15)

The post-processed detection \tilde{y} is then adjusted for the group-delay and classified into the two classes by a global estimated threshold (same threshold for all subjects), cf. FIG. (1). In the voting method, the percentage of classified REM sleep mini-epochs in each epoch is computed. If the percentage was higher than an estimated threshold, then the whole epoch was labeled as REM sleep. The estimations were obtained by including the variables in the grid-search approach as described earlier.

In an embodiment of the present invention m of the current study is to automatically detect the REM sleep stage according to the new international sleep-scoring standard from the American Academy of Sleep Medicine (AASM). According to the AASM a sleep stage epoch of 30 seconds must be scored as REM when the electroencephalography (EEG) has low amplitude with mixed frequencies (i.e. 4-7 Hz) in the frontal, central and occipital electrodes Furthermore, there should also be relatively low muscle tone in the chin. If there are no indications of another sleep stage between the REM events in the electrooculography (EOG) channels, it is assumed to be REM sleep [10]. In a previous study it was concluded, based on the data and method, that the muscle tone had little, if any, influence on the REM sleep detection. However, the method has been modified by subject-specific feature scaling, and post-processing of the classifier prediction. This turned out to improve the overall classification performance. Thus, an automatic REM sleep detector has been provided.

One embodiment of the invention may utilise a device for measuring and recording of physiological signals. The device may include, or be connected to, surface electrodes for measuring e.g. EEG, EMG, ECG and respiration frequency. These are placed according to the AASM international standard and recordings are performed. The electrodes are connected to a recording device which preferably is a portable device which makes it possible for the person to have the electrodes attach at a clinic and still take the device home for overnight recordings in the home. Recordings are preferably performed ambulant and during at least one full night and in some embodiment at a minimum of 12 hours. The processing includes analysing the bipolar EMG of the chin.

The surface detected signals are often affected by noise from slow movement and instability of the electrode-skin interface which typically can be seen in the range of 0-20 Hz. Furthermore many signals are also affected by the powerline noise (i.e. 50 Hz in Europe). To reduce the interferences all the signals are preprocessed, in one embodiment, using a band-pass filter and a notch filter. The applied band-pass filter is in one embodiment of the invention an 8th order Butterworth filter with a 3 dB cut off frequency of 20 Hz and 60 Hz respectively, and the notch filter is a 2nd order Butterworth notch filter with a 3 dB cut off frequency of 48 Hz and 52 Hz respectively.

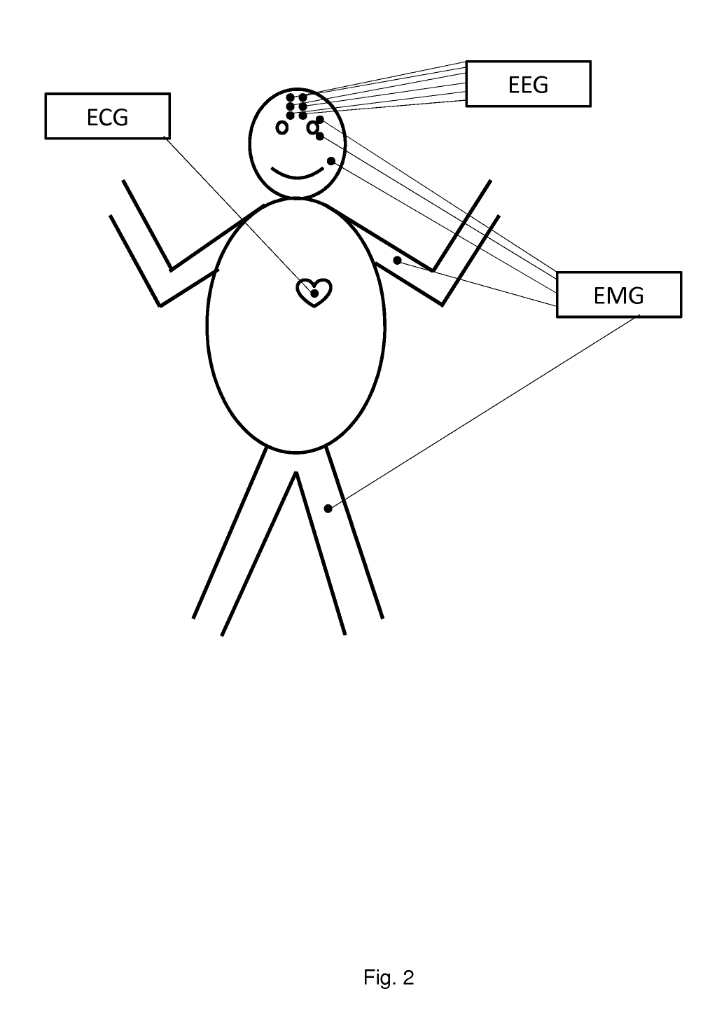

FIG. 2 schematically illustrates a person wearing a set of electrodes. The electrodes are placed as EEG, ECG and EMG electrodes. In certain embodiments not all three types of electrode positions are used. In particular embodiments not all electrodes of the EEG, EMG or ECG groups are used.

The electrodes in FIG. 2 may be positioned so as to obtain one or more of muscle activity at or near the eye, eye movement morphology, muscle activity measured from one or more body parts including limbs and head, respiration frequency, heart rate, an electroencephalographycal (EEG) signal, an electrooculographycal (EOG) signal, an eletrocardiographycal (ECG) signal and/or an electromyographycal (EMG) signal.

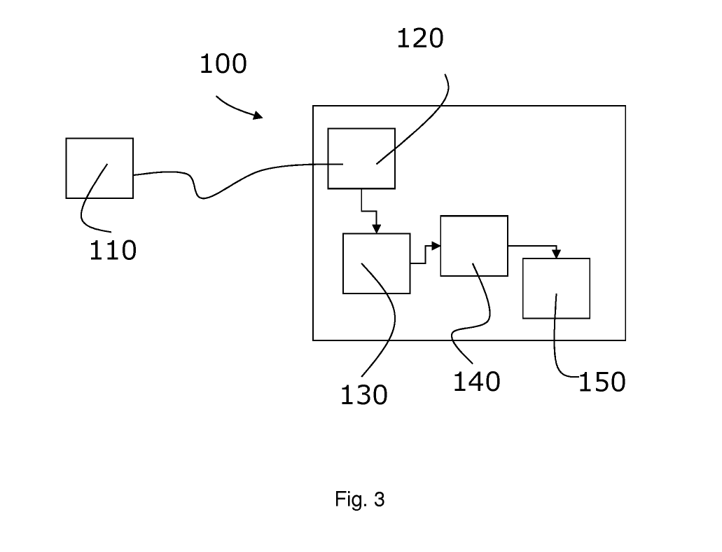

FIG. 3 is a schematic illustration of a system 100 for assisting a health care person in assessing sleep and/or wake patterns in a person. The system 100 comprises a set of electrodes 110 for recording a set of physiological signals in a time interval. These electrodes may be placed as illustrated and discussed in relation to FIG. 2. The system 100 comprises a filter 120 for filtering the set of physiological signals so as to reduce artefact and/or noise from the set of physiological signals. The filter 120 may be applied after the signals from the set of electrodes 110 are AD converted. In some embodiments the signals from the set of electrodes 110 are filtered before they are AD converted. The system 100 comprises a pattern classifier device 130 for classifying a sleep and/or wake pattern in one or more sub-time intervals of the time interval, wherein the classification is based on a signal from the set of physiological signals and wherein the sleep and/or wake pattern is classified as a first type or a second type. The pattern classifier device 130 received a signal, or group of signals from the filter 120. The system 100 comprises a motor classifier 140 for classifying a motor pattern in each of the one or more sub-time intervals based on a signal from the set of physiological signals and the sleep and/or wake type into a first motor pattern type or a second motor pattern type. The motor classifier 140 may receive the signals from both the filter 120 and the pattern classifier device 130. Alternatively the motor classifier 140 may receive a signal from the pattern classifier device 130 alone, as this may include both the sleep and/or wake type as well as the set of physiological signals. The system 100 comprises a processor 150 for computing a motor descriptor based on the motor pattern type. In some embodiments the processor 150 may be provided with the data determined by the other parts of the system 100. Each component in the system may be connected to a common memory device. The common memory device may be embodied as a RAM storage, a permanent storage such as a hard drive and a data base. Other suitable types of storage may be used.

The filter 120 is in an embodiment the filter is band-pass filter. The band-pass filter is a fourth-order Butterworth filter with a cut-off frequency of 30 Hz and 60 Hz respectively.

The invention can be implemented by means of hardware, software, firmware or any combination of these. The invention or some of the features thereof can also be implemented as software running on one or more data processors and/or digital signal processors.

The individual elements of an embodiment of the invention may be physically, functionally and logically implemented in any suitable way such as in a single unit, in a plurality of units or as part of separate functional units. The invention may be implemented in a single unit, or be both physically and functionally distributed between different units and processors.

Although the present invention has been described in connection with the specified embodiments, it should not be construed as being in any way limited to the presented examples. The scope of the present invention is to be interpreted in the light of the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

### Example 1

### iRBD Study of 48 Subjects

A total of 48 subjects (16 subjects with a diagnosis of iRBD, 16 with PLM and 16 age-matched healthy controls) from the Danish Center for Sleep Medicine, Department of Clinical Neurophysiology, Glostrup University Hospital, Denmark, were enrolled in the study (American Academy of Sleep Medicine, 2005). The subjects were divided into two groups on the basis of their diagnosis (i.e. iRBD subjects versus healthy controls and PLM subjects). The demographics of the groups are summarized in table 1.1.

TABLE 1.1
Demographics of the 48 study subjects.

| | | No subjects | Age mean ± std. |
|---|---|---|---|
| Group | Diagnosis | (♀, ♂) | [years] |
| Negative | Control | 16 (11, 5) | 56.1 ± 9.4 |
| Negative | PLM | 16 (9, 7) | 57.8 ± 10.5 |
| Positive | iRBD | 16 (3, 13) | 58.4 ± 13.5 |

The inclusion of the PLM subjects was based on the polysomnography (PSG) independently of clinical symptoms (PLM index ≧5). Individuals with other abnormalities known to affect sleep, such as sleep apnea or stridor were excluded from the study. Their diagnosis is based on respiratory abnormalities and not abnormalities in the sEMG. The controls and subjects involved were not taking any medicine that is known to affect sleep. The data gathered did not allow balancing the two groups with respect to age and gender.

### Polysomnography Montage and Data Extraction

All subjects underwent a full night PSG in accordance with the American Academy of Sleep Medicine (Iber et al., 2007). However, not all subjects were monitored with video camera (video-PSG). Most of the healthy controls were recorded ambulant. To ensure the quality of each recording, all recordings were visually inspected by specialists. Corrupted recordings in which the analysis channels became disconnected or continuously contaminated with artifacts were rejected. Sleep data were analyzed at a sampling frequency of 256 Hz. They were recorded over a period of five years, using different amplifier systems, in which the lowest cutoff frequency of the anti-aliasing filters was 70 Hz. The manual sleep scores were extracted from the sleep program Nervus and, via transformation to European Data Format (Kemp et al., 1992), converted into MATLAB format for further signal processing.

### Preprocessing

The sEMG is often contaminated with artifacts from changing electrode-skin contact, powerline noise, electrocardiography (ECG) crosstalk, or even respiration artifacts. Baseline drift and ECG crosstalk were reduced by applying a fourth-order Butterworth (BW) bandpass filter, using a zero-phase filtering approach (Gustafsson, 1996). The chosen cutoff frequencies (3 dB) were 30 Hz and 65 Hz, respectively (Merletti and Parker, 2004; Drake and Challaghan, 2006). Similarly, power-line noise was reduced with a fourth-order BW notch filter with the cutoff frequencies (3 dB) 48 Hz and 52 Hz, respectively.

### Sleep Data

The total amount of recorded epochs in percentage from light-off to light-on and related sleep parameters are summarized in table 1.2, where NREM corresponds to NREM1, NREM2 and NREM3.

TABLE 1.2
Sleep parameters of the 48 study subjects.

| | Control | PLM | iRBD |
|---|---|---|---|
| | (mean ± std.) | (mean ± std.) | (mean ± std.) |
| Wakefulness [%] | 15.5 ± 6.6 | 16.9 ± 6.7 | 19.1 ± 7.4 |
| NREM [%] | 64.6 ± 7.1 | 65.8 ± 6.3 | 63.2 ± 7.5 |
| REM [%] | 19.8 ± 5.8 | 17.4 ± 6.2 | 17.7 ± 5.5 |
| Total sleep time [min] | 418 ± 55 | 357 ± 52 | 397 ± 79 |
| REM sleep latency | 76 ± 22 | 105 ± 63 | 136 ± 93 |
| [min] | | | |
| REM sleep arousal | 12.5 ± 7.5 | 4.1 ± 3.2 | 5.0 ± 7.0 |
| index | | | |
| REM sleep PLM index | 1.5 ± 1.8 | 1.8 ± 1.8 | 7.0 ± 9.2 |
| Apnea index (AHI) | 2.6 ± 3.7 | 4.6 ± 4.6 | 4.6 ± 5.9 |

Detection of REM Sleep without Atonia

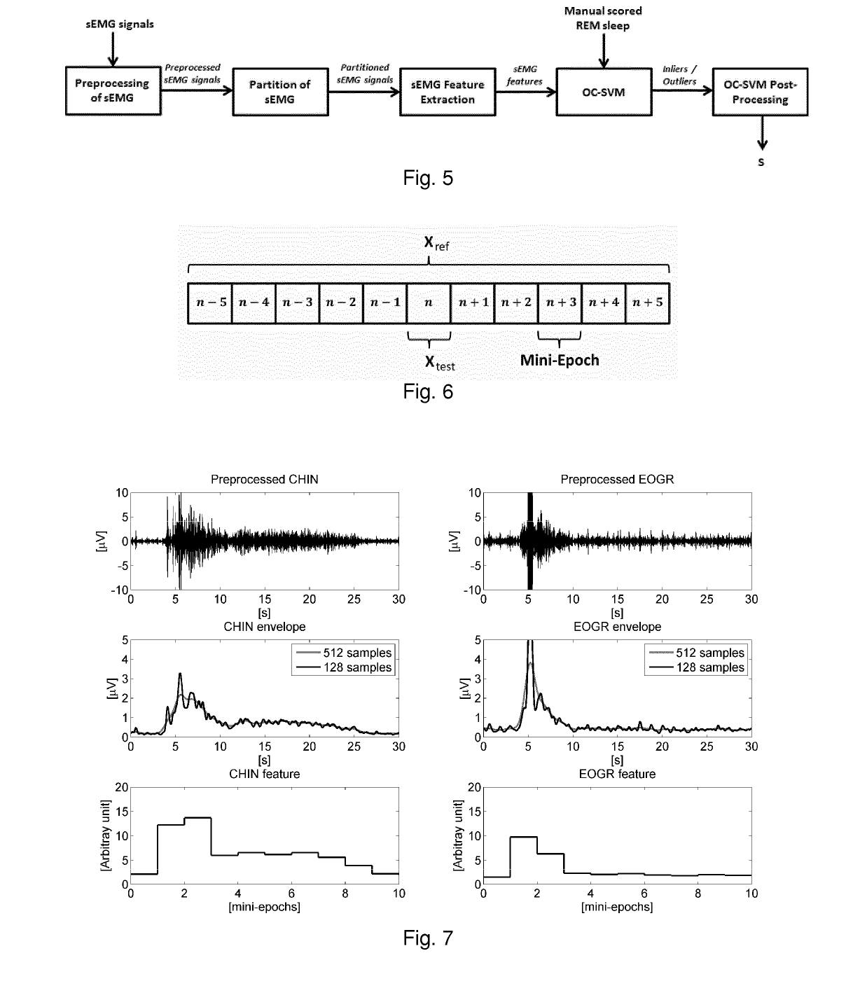

The detection of abnormal high muscle activity during REM sleep (RSWA) was constructed as illustrated in the flowchart in FIG. 5. Each block will be addressed in the following sections.

FIG. 5 is a flowchart of the proposed RSWA detector. Five sEMG channels were examined, corresponding to the left and right EOG channels (EOGL, EOGR), the submentalis (CHIN) and the left and right anterior tibialis channel (TIBL, TIBR). First, the signals were preprocessed and partitioned. Then the muscle activity features were computed. Muscle activity is thought to be outliers relative to the background sEMG activity during REM sleep. Therefore, the number of partitions that have been classified as outliers relative to the total REM sleep is used as a muscle activity score, denoted S.

Envelope of the sEMG Signals

As an alternative to the known sEMG features, such as root-mean-square, sEMG can be described by its envelope curve. In this study the envelope curve is obtained by smoothing the full-wave-rectified preprocessed sEMG signals. A normalized Blackman window with a duration of 128 samples, according to (Gustafsson, 1996), was employed. Other durations (64, 256 and 512 samples), and windows (rectangular, Hanning and Kaiser) were also cross-validated. A duration of 128 performed best with the present data. The rectangular window performed the worst, whereas the other windows performed similarly. Illustration of the envelope can be found in FIG. 7.

### Partition of Data

The envelope from a given subject was partitioned into mini-epochs of 3-second duration. This duration is widely used in the sleep research community (Iber et al., 2007). One characterizing feature was extracted from each mini-epoch. This was conducted on a mini-epoch basis, using a sliding window as illustrated in FIG. 6. The reference window in FIG. 6 consists of 11 mini-epochs, while the central test window consists of 1 mini-epoch. The step size is one mini-epoch.

Five mini-epochs on either side of the center (n) mini-epoch (xₜₑₛₜ) were included in the reference window (x_{ref}). The total reference window duration was 11 mini-epochs (33 seconds) and the duration of the test window was 1 mini-epoch (3 seconds). The step size of the sliding window was 1 mini-epoch (3 seconds). Other reference window durations were also cross-validated. However, increasing the total duration of the reference window (>33 seconds) did not change the outcome. Furthermore, the partitioned envelope signal from light-off to light-on was expanded at the beginning and end. This was accomplished by repeating the five first mini-epochs at the beginning and the final five mini-epochs at the end.

### Feature Extraction

For diagnosing iRBD (Frauscher et al., 2012) recommended that any type of sEMG activity, whether it consists of tonic, phasic or a combination of the two should be used to quantify the muscle activity. Therefore, a simple “on-off” sEMG feature is proposed. The sEMG has to be analyzed relatively, and cannot be directly compared between muscles or subjects. Therefore, the activity in the test window (FIG. 2) was compared with the reference window according to:

$$G ( n ) = \frac{mean ( {x}_{test} ( n ) )}{min ( {x}_{ref} ( n ) ) + ɛ} \\ G ( n ) = \frac{mean ( {x}_{test} ( n ) )}{min ( {x}_{ref} ( n ) ) + ɛ} \qquad ( 1.1 )$$

Where n is the mini-epoch index and xₜₑₛₜ and x_{ref} are the envelopes of the test and reference window, respectively. In other words, the feature (1.1) compares the mean of the envelopes in the test window with the minimum (assumed atonia) of the envelopes in the reference window. To ensure G<∞, the minimum was not allowed to be zero. This was ensured by adding ε=10⁻⁵ to the denominator. Other values of ε were also cross-validated, but only higher values affected the outcome. Using the minimum as a baseline measure ignores any activity in the reference window. Other baseline measures may be tested, such as the mean and median of the reference window. A high level of activity in the reference window tends to suppress the activity in the test window.

FIG. 7 shows the muscle feature extraction process using the CHIN and EOGR channels from the same REM sleep epoch (iRBD subject). Two Blackman window durations are shown, where black corresponds to a duration of 128 samples (0.5 second), while red corresponds to 512 samples (2 seconds).

The relative measure between the reference window and the test window is denoted by G. It is constant and low when there is no muscle activity, but increases with muscle activity. The relative measure G was computed from light-off to light-on from all the five sEMG channels and from each of the 48 subjects.

### Outlier Detection

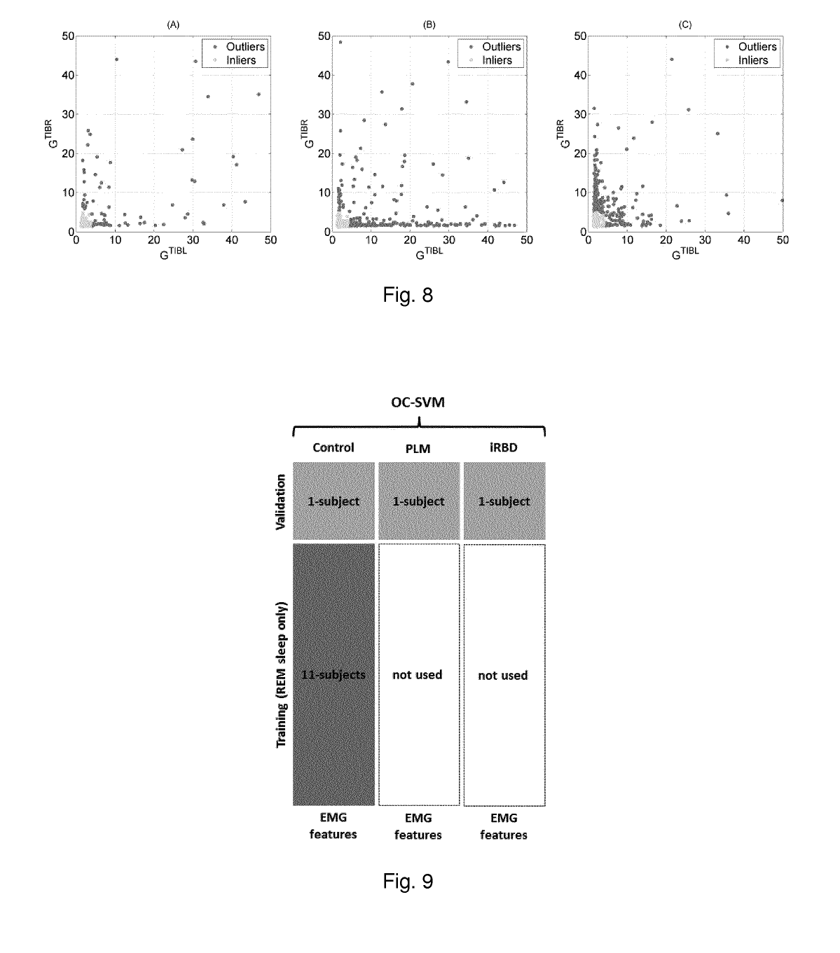

The objective of the outlier detector, also called a one-class support vector machine (OC-SVM) classifier, is to classify (unsupervised) the feature samples (G) into two classes: inliers and outliers. It is assumed that the inlier class corresponds to normal REM sleep mini-epochs with atonia, while the outlier class corresponds to RSWA mini-epochs. FIG. 8 shows an example of scatterplots of given subjects during REM sleep from one healthy control (A), one PLM (B) and one iRBD subject (C). Green and red dots indicate inliers (atonia) and outliers (RSWA), respectively. The sleep stage, in this case REM sleep, was obtained by selecting the mini-epochs that were scored as REM sleep using the manually scored hypnogram.

Each dot in FIG. 8 corresponds to one feature value computed by (1.1) using the TIBL and TIBR channels respectively. The green and red dots correspond to classified inliers and outliers, respectively. The inlier and outlier labels were obtained by the OC-SVM classifier. It is noted how the inliers in all three plots in FIG. 8 are located in a small region in the lower left corner that captures most of the data points, whereas the outliers occur in a larger region that captures fewer data points. It is assumed that the OC-SVM classifier has enclosed, or captured the atonia examples (green). Examples that fall outside, due to loss of atonia (RSWA), are assumed to be outliers (red). The relative number of outliers to the inliers was used as a quantitative measure of muscle activity. The challenge is to adjust the OC-SVM classifier so it correctly encloses the atonia examples. This will be addressed in the following sections.

### The One-Class Support Vector Machine

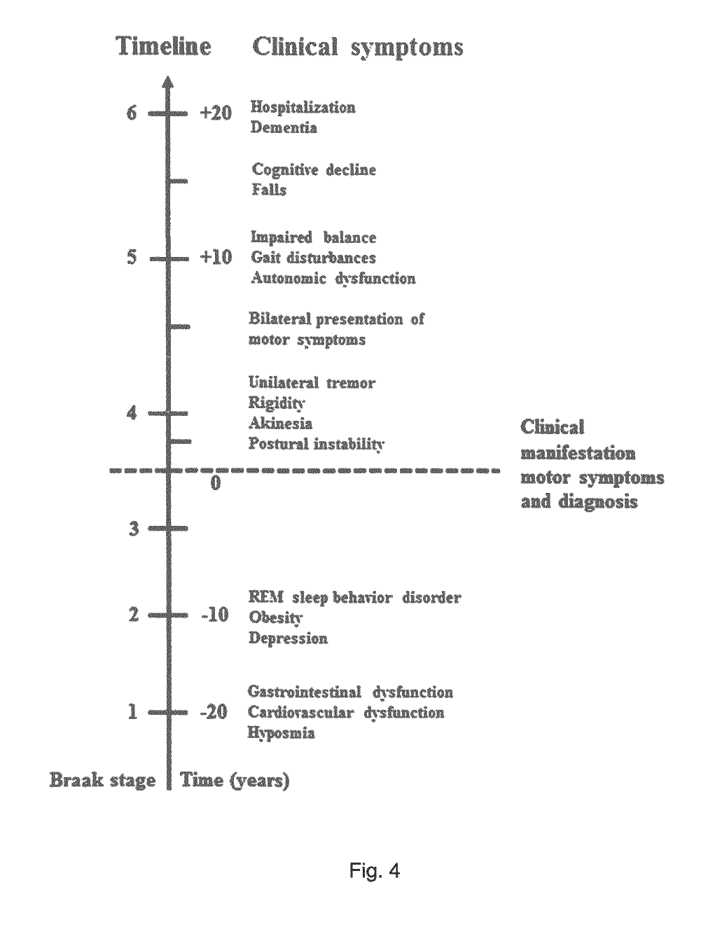

The two-class (binary) support vector machine (BC-SVM) is a relatively new and widely used supervised-learning algorithm, originally introduced by (Boser et al., 1992; Cortes and Vapnik, 1995). The OC-SVM is an extension of the original BC-SVM learning algorithm (Schölkopf et al., 2001; Shawe-Taylor and Cristianini, 2004). The OC-SVM has been successfully applied in various fields, such as fraud detection, text document classification and medical diagnosis. In contrast to the SVM, which finds the discriminative boundary between two classes, the OC-SVM finds the smallest possible boundary that encloses most of the target data. This may be determined in the absence of any anti-target data. The OC-SVM algorithm returns a function ƒ(x) that takes the value +1 in a “small” region that captures most of the data points, and a value of −1 everywhere else (FIG. 4), where x is the feature computed by (1). With respect to this study, a set of training vectors xᵢε [symbol] ⁿ, i=1, . . . , l computed from the healthy control subjects are available. The OC-SVM solves the optimization problem:

$$\begin{matrix}{min}_{w , ρ , ξ} \frac{1}{2} {‖ w ‖}^{2} + \frac{1}{v N} {\sum }_{i = 1}^{N} {ξ}_{i} - ρ \\ subject to & ( 1.2 ) \\ ( w \cdot φ ( {x}_{i} ) ) \geq ρ - {ξ}_{i} \\ and & ( 1.3 ) \\ {ξ}_{i} \geq 0 , \\ i = 1 , … , N & ( 1.4 )\end{matrix}$$

where w and ρ are the weights and offset respectively, both of which have to be estimated. ξᵢ is the slack variable, which specifies the amount of misplacement each data point contributes with. To avoid overfitting the parameter νε[0,1] is introduced. It characterizes the fraction of outliers (i.e. proportion of data points for which the function ƒ(x) takes the value −1). In this study the Radial Basic Function (RBF) kernel was selected, which maps x onto an inner product space (feature space), such that the dot product in this feature space can be computed by evaluating the kernel defined by:

K(xᵢ,xₖ)=φ(xᵢ)·φ(xₖ)=e^{-γ∥x}^{ᵢ}^{-x}^{ₖ}^{∥}^{²} (1.5)

Once the separating hyperplane has been identified, it can be used to classify unseen feature points by testing on which side of the hyperplane the point lies:

ƒ(x)=sgn((w·φ(x))−ρ) (1.6)

where sgn is the sign function. In summary, the classifier output can take one of two values ƒ(x)ε(−1,1) corresponding to each class. In this study, a value of ‘−1’ corresponds to an outlier (RSWA), while ‘+1’ corresponds to an inlier, i.e. REM sleep with atonia. The tested OC-SVM parameters were ν=0.01, 0.02 . . . , 0.1 and γ=2⁻⁸, 2⁻⁷, . . . , 2⁸. The experiments presented were performed using a freely available MATLAB implementation of the SVM classifier (LIBSVM), which was chosen for its good performance and ability to handle large amounts of data, as was necessary in this study (Chang and Lin, 2011). Furthermore, a well written user guide was also available (Hsu et al., 2011).

### Classification

The optimal adjustable parameters were found by cross-validating different parameter combinations. This was achieved by conducting the cross-validation on a subset of the 48 collected subjects; in this case the first 36 collected subjects (12 subjects from each of the three groups). The method was then tested on unseen data, i.e. the remaining 12 subjects (four subjects from each of the three groups). The parameter optimization procedure is summarized in FIG. 9 which illustrates the results of a leave-one-subject-out-cross-validation. First training on 11 healthy controls, and then validating on one healthy subject that was not included in the training. Furthermore, validation is also provided on one random selected iRBD subject and one random selected PLM subject. This process is repeated further 11 times, each time leaving a different healthy control in turn as the validation subject. None of the iRBD and PLM subjects were validated more than once.

All the above mentioned adjustable parameters were found by cross-validating different parameter combinations. To do this, a simple grid-search approach was used, in combination with the leave-one-subject-out cross-validation scheme. It is assumed that healthy controls experience few, if any, muscle activations during REM sleep.

Therefore, inspired from (Mourão-Miranda et al., 2011), the OC-SVM classifier was trained using only healthy controls. The REM sleep features from the healthy controls were selected from the manual scored hypnogram. Since the feature samples from each subject may be correlated, a fold consists of whole subjects. A single fold (1 healthy control) was held out for validation, while the remaining 11 folds (11 healthy controls) were used for training. The trained OC-SVM was then validated on the healthy control left out and one randomly selected iRBD subject and one randomly selected PLM subject. This procedure was repeated further 11 times, leaving out each healthy control in turn as the validation subject, which was matched to a different iRBD and PLM subject each time. None of the healthy controls, iRBD or PLM subjects were validated more than once. The OC-SVM output (inliers/outliers) was then post-processed. The amount of outliers, with respect to the amount of inliers in each validation subject, was used as a quantitative measure of the muscle activity. The muscle score in percentage is given by:

$$S = \frac{\# outliers}{\# outliers + \# liers} \cdot 100 \qquad ( 1.7 )$$

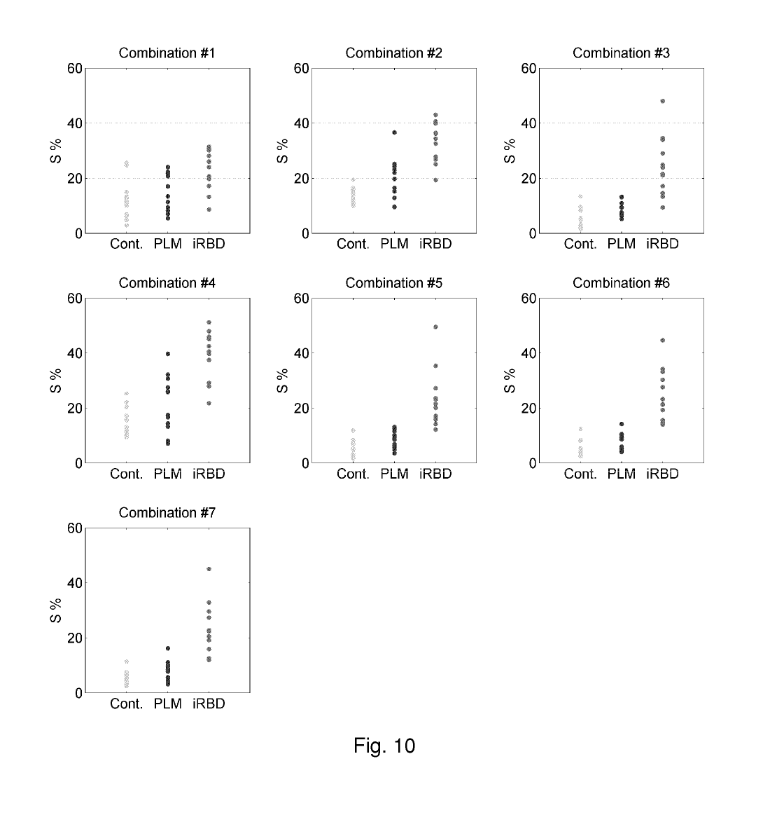

The three muscle scores (S_{control}, S_{iRBD}, S_{PLM}) from the 12 validations, were then merged yielding a total of 36 score values (Forman and Scholz, 2010); i.e. one muscle score from each validation subject. An example of muscle scores can be found in FIG. 10. The discriminability was measured by the area under the receiver operating characteristic (ROC) curve (AUC), in which the iRBD scores were considered to be the positive instances (positive group), and the healthy controls and PLM scores were taken as the negative instances (negative group). This is also described in table 1.1. The above mentioned cross-validation scheme was repeated 25 times, and the parameter combination with the highest average AUC (AUC_{μ}) was selected. This will reduce the influence of the random selected PLM and iRBD subjects in the validation folds. The parameter combination with the highest AUC_{μ} was then chosen, and used for training using the same 36 validation subjects and subsequently applied and tested on the 12 unseen subjects.

### Results

Seven experiments were conducted, involving different combinations of the channels. The combinations are defined in table 1.3, where a cross (‘x’) indicates an active channel, and a blank indicates an inactive channel.

TABLE 1.3
Validation and test results. μ and σ are the mean and standard deviation
across the 25 runs of the validation scheme, respectively.

| | | | | | | SVM | | | | |
|---|---|---|---|---|---|---|---|---|---|---|
| | Channels | | | | | parameters | | Validation | | Test |
| Comb. No | CHIN | EOGL | EOGR | TIBL | TIBR | ν | γ_{OC} | AUC_{μ} | AUC_{σ} | AUC |
| 1 | x | | | | | 0.03 | 2⁴ | 0.834 | 0.016 | 0.906 |
| 2 | | x | x | | | 0.04 | 2⁵ | 0.946 | 0.009 | 0.906 |
| 3 | | | | x | x | 0.05 | 2⁻³ | 0.974 | 0.007 | 1.000 |
| 4 | x | x | x | | | 0.02 | 2⁴ | 0.933 | 0.007 | 0.875 |
| 5 | x | | | x | x | 0.05 | 2⁻⁴ | 0.993 | 0.003 | 1.000 |
| 6 | | x | x | x | x | 0.05 | 2⁻⁵ | 0.993 | 0.003 | 1.000 |
| 7 | x | x | x | x | x | 0.05 | 2⁻⁵ | 0.989 | 0.003 | 1.000 |

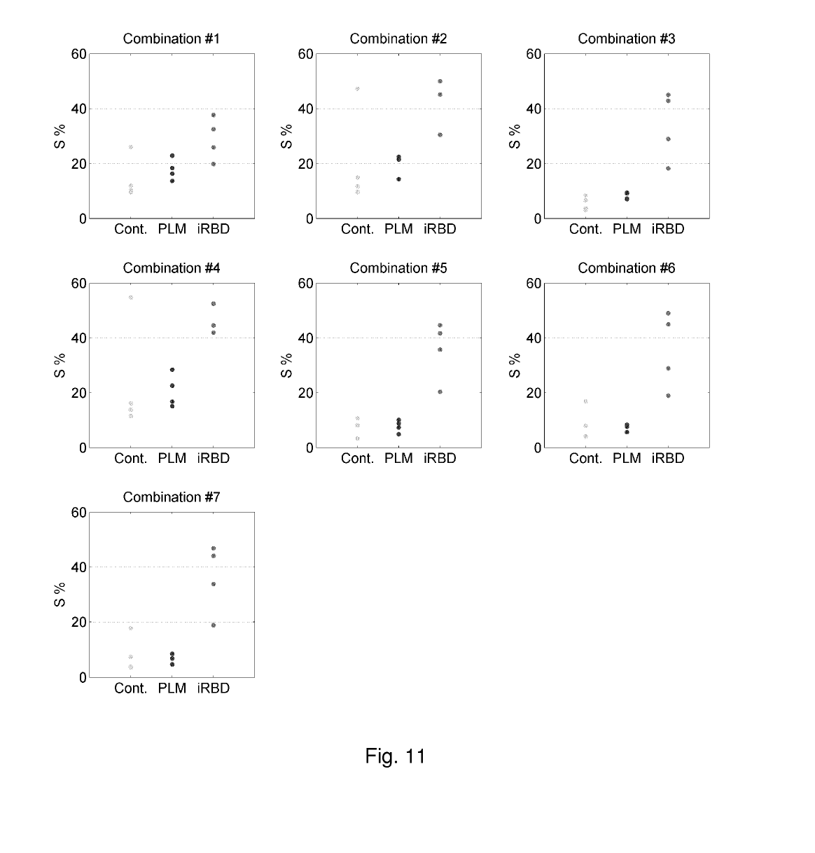

An example of the muscle scores of the seven combinations, using the parameters from table 1.3, is shown in FIG. 10 and FIG. 11. FIG. 10 shows validation muscle scores during REM sleep of one random selected run (36 subjects). Seven combinations have been tested during REM sleep. These are defined in table 1.3. FIG. 11 shows test muscle scores during REM sleep (12 subjects). Seven combinations have been tested during REM sleep. These are defined in table 1.3.

### Discussion

This study validates one embodiment of the present invention relating to the detection of RSWA, which is assumed to be an early symptom for PD. This was achieved by investigating the combinations of five sEMG channels (CHIN, TIBL, TIBR, EOGL and EOGR). Typically, periodic limb movements (PLMs) of sleep appear in NREM sleep, and are very infrequent in the normal/non-diseased population during REM sleep, whereas PLMs during REM sleep are very common in iRBD subjects (American Academy of Sleep Medicine, 2005). To gain a better understanding of PLMs during REM sleep the present study also included subjects diagnosed with PLM disorder. Therefore, 48 subjects were enrolled, 16 subjects each with a diagnosis of iRBD and of PLM, and 16 age-matched healthy controls. The subjects were divided into two groups on the basis of their diagnosis (i.e. iRBD subjects versus healthy controls and PLM subjects).

Only a few computerized (semi-automatic) prior art algorithms have focused on iRBD, particularly on early detection of PD. (Burns et al., 2007; Kempfner et al., 2010; Ferri et al., 2012) none of the enrolled subjects were diagnosed with iRBD. They focused on neurodegenerative subjects, some with RBD, using the chin sEMG electrode. However, (Mayer et al., 2008) enrolled 48 subjects, 21 of whom were diagnosed with iRBD. They sought to quantify the muscle activity into short- and long-duration movements. Both types were significantly higher in subjects with RBD. (Shokrollahi et al., 2009a, 2009b) tried to discriminate muscle activity during REM sleep on subjects with RSWA using the chin sEMG electrode. They obtained promising results with respect to classifying abnormal partitions, but only in four healthy controls and four subjects with RSWA. None of the above mentioned studies have included the EOG as an additional sEMG channel, which in this study demonstrated high discriminability, and only (Mayer et al., 2008) included other sEMG electrodes than chin.

During REM sleep the TIB (TIBR and TIBL) was discriminatively powerful, and on its own was able to separate the two groups almost perfectly with an AUC of 0.974. These results were confirmed by testing the 12 unseen subjects, where all subjects were classified correctly. When combining TIB with CHIN or EOG the separability increases even further. This makes the EOG an attractive channel, especially with respect to noisy PSG recordings, since it may be used as an alternative to CHIN in situations where CHIN is heavily affected by artifacts, or has fallen off. In this study the EOG performed better than CHIN. These results differ from the Sleep Innsbruck Barcelona (SINBAR) group. The SINBAR group visually found that the combination of the mentalis muscle and the flexor digitorum superficialis muscle was superior in detecting RSWA (Frauscher et al., 2012).

Apart from noise, outliers may also be related to other sleep phenomena, such as arousals and apnea, which, during REM sleep, may be associated with increased muscle activity and affect the muscle score (Iber et al., 2007). However, apnea is not a movement disorder, and is diagnosed based on the respiratory pattern. They were therefore excluded in this study. The arousals were ignored in this study, assuming their influence on the muscle score would be low. This assumption seems to be confirmed by this study, since the REM sleep arousal index was highest in the healthy group (table 1.2). However, if arousals should be addressed, the issue of when an arousal ends must be discussed. Sometimes the sEMG returns to atonia after the arousal has official ended. In other words, the arousals must be scored in such way that the scoring includes the increased sEMG activity. This was not included in this study, since the arousals were scored according to the AASM.

In the present study the interrater variability of the sleep scores may be reduced by introducing a computerized REM sleep-stage detector, such as the one proposed by Kempfner et al. in 2012) employing standard EEG and EOG channels, providing correct detection of REM sleep with a sensitivity and specificity of 0.94 and 0.96 in 8 iRBD subjects and 8 healthy controls.

### REFERENCES

American Academy of Sleep Medicine. International classification of sleep disorders: diagnostic and coding manual. 2nd ed. Westchester, Ill.: AASM, 2005.
Boeve B, Silber M, Ferman T, Lucas J, Parisi J. Association of REM sleep behavior disorder and neurodegenerative disease may reflect an underlying synucleinopathy. Mov Disord 16: 622-630, 2001.
Boser B, Guyon M, Vapnik V. A training algorithm for optimal margin classifiers. In: Proceedings of the 5th Annual ACM Workshop on Computational Learning Theory 1992; 144-152.
Burns J, Consens F, Little R, Angell K, Gilman S, Chervin R. EMG variance during polysomnography as an assessment for REM sleep behavior disorder. Sleep 2007; 30: 265-271.
Cortes C, Vapnik V. Support-vector network. Machine Learning 1995; 20: 273-297.
Drake J, Challaghan J. Elimination of electrocardiogram contamination from electromyogram signals: An evaluation of currently used removal techniques. J Electromyogr Kinesiol. 2006; 16: 175-187.
Ferri R, Fulda S, Cosentino F, Pizza F, Plazzi G. A preliminary quantitative analysis of REM sleep chin EMG in Parkinson's disease with or without REM sleep behavior disorder. Sleep Med 2012; 13: 707-713.
Forman G, Scholz M. Apples-to-apples in cross-validation studies: pitfalls in classifier performance measurement. ACM SIGKDD Explorations Newsletter 2010; 12: 49-57.
Frauscher B, Iranzo A, Gaig C, Gschliesser V, Guaita M, Raffelseder V, Ehrmann L, Sola N, Salamero M, Tolosa E, Poewe W, Santamaria J, Högl B. Normative EMG values during REM sleep for the diagnosis of REM sleep behavior disorder. Sleep 2012; 35: 835-847.
Gustafsson F. Determining the initial states in forward-backward filtering. IEEE Transactions on Signal Processing 1996; 44:988-992.
Iber C, Ancoli-Israel S, Chesson A, Quan S. The AASM Manual for the scoring of sleep and associated events: rules, terminology and technical specification. Westchester, Ill.: AASM, 2007.
Iranzo A, Molinuevo J, Santamaria J, Serradell M, Marti J, Valldeoriola F, Tolosa E. Rapid-eye-movement sleep behaviour disorder as an early marker for a neurodegenerative disorder: a descriptive study. Lancet Neurol 2006; 5: 572-577.
Kemp B, Varrib A, Rosac A, Nielsend K, Gaded J. A simple format for exchange of digitized polygraphic recordings. Electroencephalogr Clin Neurophysiol 1992; 82: 391-393.
Kempfner J, Jennum P, Nikolic M, Christensen J, Sorensen H. Automatic detection of REM sleep in subjects without atonia. In: Conf Proc IEEE Eng Med Biol Soc. 2012; 4242-4245.
Kempfner J, Sorensen G, Sorensen H, Jennum P. Automatic REM sleep detection associated with idiopathic REM sleep behavior disorder. In: Conf Proc IEEE Eng Med Biol Soc 2011; 6063-6066.
Kempfner J, Sorensen G, Zoetmulder M, Jennum P, Sorensen H. REM Behaviour disorder detection associated with neurodegenerative diseases. In: Conf Proc IEEE Eng Med Biol Soc 2010; 5093-5096.
Mayer G, Kesper K, Ploch T, Canisius S, Penzel T, Oertel W, Stiasny-Kolster K. Quantification of tonic and phasic muscle activity in REM sleep behavior disorder. J Clin Neurophysiol 2008; 25: 48-55.
McCarter S, St Louis E, Boeve B. REM sleep behavior disorder and REM sleep without atonia as an early manifestation of degenerative neurological disease. Curr Neurol Neurosci Rep 2012; 12: 182-192.
Merletti R, Parker P. Electromyography—Physiology, Engineering, and non-invasive application. 1st ed. New Jersey: Wiley-IEEE Press, 2004.
Mourão-Miranda J, Hardoon D, Hahn T, Marquand A, Williams S, Shawe-Taylor J, Brammer M. Patient classification as an outlier detection problem: an application of the one-class support vector machine. Neuroimage 2011; 58: 793-804.
Postuma R, Gagnon J, Rompré S, Montplaisir J. Severity of REM atonia loss in idiopathic REM sleep behavior disorder predicts Parkinson disease. Neurology 2010; 74: 239-244.
Postuma R, Gagnon J, Vendette M, Fantini M, Massicotte-Marquez, J. Montplaisir J. Quantifying the risk of neurodegenerative disease in idiopathic REM sleep behavior disorder. Neurology 2009; 72: 1296-1300.
Schenck C, Bundlie S, Mahowald M. Delayed emergence of a parkinsonian disorder in 38% of 29 older men initially diagnosed with idiopathic rapid eye movement sleep behaviour disorder. Neurology 1996; 46: 388-393.
Schölkopf B, Platt J, Shawe-Taylor J, Smola A, Williamson R. Estimating the support of a high-dimensional distribution. Journal Neural Computation 2001; 13: 1443-1471.
Shawe-Taylor J, Cristianini N. Kernel methods for pattern analysis. Cambridge University Press, 2004.
Shokrollahi M, Krishnan S, Jewell D, Murray B. Analysis of the electromyogram of rapid eye movement sleep using wavelet techniques. In: Conf Proc IEEE Eng Med Biol Soc 2009a; 2659-2662.
Shokrollahi M, Krishnan S, Jewell D, Murray B. Autoregressive and cepstral analysis of electromyogram in rapid movement sleep. In: WC IFMBE Proceedings 2009b; 1580-1583.

1. A method for detection of abnormal motor activity during REM sleep comprising the steps of:
a. performing polysomnographic recordings of a sleeping subject, thereby obtaining one or more electromyography (EMG) derivations, preferably surface EMG recordings, and one or more EEG derivations, and/or one or more electrooculargraphy (EOG) derivations,
b. detecting one or more REM sleep stages, preferably based on the one or more EEG and/or EOG derivations,
c. determining the level of muscle activity during the one or more REM sleep stages based on the one or more EMG derivations,
wherein a subject having an increased level of muscle activity during REM sleep compared to one or more normal subjects has abnormal motor activity during REM sleep.

2. The method according to claim 1, wherein said one or more electromyography (EMG) derivations are derived from a CHIN EMG electrode only.

3. The method according to claim 1, wherein said one or more electromyography (EMG) derivations are derived from eye movements only.

4. The method according to claim 1, wherein said one or more electromyography (EMG) derivations are derived from eye movement and a CHIN EMG electrode only.

5. The method according to any of preceding claims 3 to 4, wherein the eye movements are recorded by means of EOG electrodes, preferably an EOG-L and an EOG-R electrode.

6. The method according to any of preceding claims, wherein said one or more electromyography (EMG) derivations at least are derived from eye movements, a CHIN EMG electrode and leg movements, such as muscle activity in tibialis.

7. The method according to any of preceding claims, further comprising the step of applying a filter to the EMG and EEG and/or EOG signals, so as to reduce artefact and/or noise from the set of physiological signals.

8. The method according to any of preceding claims, wherein the level of muscle activity determined during REM sleep is based on eye movements only, and/or submentalis movements only and/or eye movements and submentalis movements only.

9. The method according to any of preceding claims, further comprising the step of classifying a motor activity, such as a muscle activity, in a plurality of time intervals (mini epocs) of the REM sleep stages, based on one or more of the EMG derivations, into a first motor activity type, such a REM sleep with atonia, or a second motor activity type, such as REM sleep without atonia (RSWA).

10. The method according to any of preceding claim 9, wherein an increased level of muscle activity during REM sleep is detected based on the classification of the time intervals during REM sleep stages into said first and second motor activity types.

11. The method according to any of preceding claims 9 to 10, wherein an increased level of muscle activity during REM sleep is detected based on the number of time intervals characterized as first and/or second motor activity types.

12. The method according to any of preceding claims 9 to 11, wherein an increased level of muscle activity during REM sleep is detected based on the number of time intervals characterized as first motor activity type relative to the number of time intervals characterized as second motor activity type.

13. The method according to any of preceding claims 9 to 12, wherein a single motor activity score, such as a single number, is computed for the subject based on the classification of the time intervals during REM sleep into first and second motor activity types and wherein an abnormal motor activity is detected for said subject based on said single score.

14. The method according to any of preceding claim 13, wherein said motor activity score is based on the number of time intervals characterized as first motor activity type relative to the number of time intervals characterized as second motor activity type.

15. The method according to any of preceding claims 9 to 14, wherein the duration of each of said time intervals is between 1 and 60 seconds, or between 1 and 30 seconds, or between 1 and 10 seconds, such as 1, 2, 4, 5, 6, 7, 8, 9, or 10 seconds, preferably 3 seconds.

16. The method according to any of preceding claims 9 to 15, wherein the classification is based on a supervised learning model, such as a support vector machine algorithm, such as the one-class support vector machine (OC-SVM) classifier.

17. The method according to any of preceding claims 9 to 16, wherein the classification is based on outlier detection, wherein muscle activity during REM sleep is defined as being outlier, or a predefined muscle activity during REM sleep is defined as being outlier, or abnormal muscle activity during REM sleep is defined as being outlier.

18. The method according to any of preceding claims, wherein an increased level of muscle activity during REM sleep is increased by about a factor 1.5 or more compared to control, for example about a factor 2 or more, or about a factor 3 or more, or about a factor 4 or more, or about a factor 5 or more.

19. The method according to any of preceding claims, wherein multiple filters are applied to the EMG data.

20. The method according to claim 19, wherein the filter is a band-pass filter, such as a fourth-order Butterworth filter with a cut-off frequency such as 30 Hz and 60 Hz respectively.

21. The method according to claim 19, wherein the filter is a notch filter, such as a fourth-order Butterworth notch-filter with a cut-off frequency.

22. The method according to any of the preceding claims, wherein the detection and determination of abnormal motor activity during REM sleep is fully automated.

23. The method according to any of the preceding claims, wherein the detection and determination of abnormal motor activity during REM sleep does not involve manual analysis of the EEG derivations by a sleep expert.

24. The method according to any of the preceding claims, wherein the level of muscle activity measured during REM sleep is in comparison to the level of muscle activity during REM sleep in a group of healthy subjects or to a previous measurement of the level of muscle activity during REM sleep in the same subject.

25. A method for identifying a subject having an increased risk of developing a synucleinopathy comprising detecting abnormal motor activity during REM sleep according to any of the preceding claims, wherein a subject having an abnormal motor activity during REM sleep has an increased risk of developing a synucleinopathy.

26. The method according to claim 25, wherein the subject is identified before clinical onset of the synucleinopathy.

27. The method according to any of the preceding claims 25 to 26, wherein the synucleinopathy is selected from Parkinson's disease, Multiple System Atrophy and Dementia with Lewy Bodies.

28. The method according to claim 25, wherein the synucleinopathy is Parkinson's disease.

29. The method according to claim 28, wherein the subject is identified before manifestation of one or more motor symptoms selected from the group consisting of tremor, rigidity, akinesia and postural instability.

30. The method according to any of the preceding claims 25 to 29, wherein the subject is identified before substantial neurodegeneration has occurred.

31. A system for detection of abnormal motor activity during REM sleep of a subject comprising,
sets of EMG and EEG and/or EOG electrodes for recording a dataset of polysomnographic signals of the subject while sleeping, and
a processing unit configured for
detecting one or more REM sleep stages, preferably based on at least a part of said dataset of polysomnographic signals,
determining the level of muscle activity during the one or more REM sleep stages based on EMG data in said dataset, and
determining whether the subject is having an increased level of muscle activity during REM sleep compared to one or more normal subjects.

32. The system according to claim 31, wherein said sets of electrodes comprises a single EMG electrode only in the form of a CHIN electrode.

33. The system according to claim 31, wherein said sets of electrodes comprises two EMG electrodes only in the form two electrodes for measure eye movement of each eye, such as an EOG-L and an EOG-R electrode.

34. The system according to claim 31, wherein said sets of electrodes comprises three EMG electrodes only in the form two electrodes for measure eye movement of each eye and one CHIN electrode.

35. The system according to claim 31, wherein said sets of electrodes comprises EMG electrodes for the eyes, legs and a CHIN electrode.

36. The system according to any of preceding claims 31 to 35, wherein the eye movements are recorded by means of EOG electrodes, preferably an EOG-L and an EOG-R electrode.

37. The system according to any of preceding claims 31 to 36, further comprising a filter adapted to reduce artefact and/or noise from the dataset.

38. The system according to any of preceding claims 31 to 37, further comprising means for carrying out the method of any of claims 1 to 30

39. A method for assessing sleep and/or wake patterns in a person, the method comprising:
recording a set of physiological signals in a time interval,
applying a filter to the set of physiological signals so as to reduce artefact and/or noise from the set of physiological signals,
classifying a sleep and/or wake pattern in one or more sub-time intervals of the time interval, wherein the classification is based on a signal from the set of physiological signals and wherein the sleep and/or wake pattern is classified as a first type, such as REM sleep, or a second type, such as NREM sleep,
classifying a motor pattern in each of the one or more sub-time intervals based on a signal from the set of physiological signals and the sleep and/or wake type into a first motor pattern type, such as normal muscle activity during REM sleep, or a second motor pattern type, such as increased muscle activity during REM sleep, and
computing a motor descriptor, such as a motor descriptor relating to rapid eye movement Sleep Behavior Disorder, based on the motor pattern type.

40. The method according to claim 1 wherein the set of physiological signals are a combination of one or more of the following physiological signals
muscle activity at or near the eye
eye movement morphology
muscle activity measured from one or more body parts including limbs and head
respiration frequency
heart rate
an electroencephalographycal (EEG) signal
an electrooculographycal (EOG) signal
an eletrocardiographycal (ECG) signal and/or
an electromyographycal (EMG) signal

41. The method according to claims 39 to 40 wherein the classification of the sleep stage, in one or more sub-time intervals based on a signal from the set of physiological signals, is based on one or more of the following classification methods: Linear and/or nonlinear classifiers including
Neural Network
Support Vector Machine
k-Nearest Neighbour and/or
Bayes Classifiers

42. The method according to claims 39 to 41, wherein the physiological signal is an electrophysiological signal.

43. The method according to any of preceding claims 39 to 42, wherein a sleep pattern is selected from the group of: REM sleep or non-REM sleep.

44. The method according to claims 39 to 43, wherein multiple filters are applied.

45. The method according to claims 39 to 44, wherein the filter is a band-pass filter.

46. The method according to claim 45, wherein the band-pass filter is a fourth-order Butterworth filter with a cut-off frequency such as 30 Hz and 60 Hz respectively.

47. The method according to claims 39 to 44, wherein the filter is a notch filter

48. The method according to claim 47 wherein the filter is a fourth-order Butterworth notch-filter with a cut-off frequency.

49. A system for assisting a health care person in assessing sleep and/or wake patterns in a person, the system comprising:
a set of electrodes for recording a set of physiological signals in a time interval,
a filter for filtering the set of physiological signals so as to reduce artefact and/or noise from the set of physiological signals,
a pattern classifier device for classifying a sleep and/or wake pattern in one or more sub-time intervals of the time interval, wherein the classification is based on a signal from the set of physiological signals and wherein the sleep and/or wake pattern is classified as a first type or a second type,
a motor classifier for classifying a motor pattern in each of the one or more sub-time intervals based on a signal from the set of physiological signals and the sleep and/or wake type into a first motor pattern type or a second motor pattern type, and
a processor for computing a motor descriptor based on the motor pattern type.

50. The system according to claim 49, wherein the set of electrodes are arranged to be positioned so as to obtain a signal characterizing one or more of:
muscle activity at or near the eye,
eye movement morphology,
muscle activity measured from one or more body parts including limbs and head,
respiration frequency,
heart rate,
an electroencephalographycal (EEG) signal,
an electrooculographycal (EOG) signal,
an eletrocardiographycal (ECG) signal and/or
an electromyographycal (EMG) signal.

51. The system according to claim 49, wherein the filter is a notch filter.

52. The system according to claim 49, wherein the filter is a band-pass filter.

53. The system according to claim 52, wherein the band-pass filter is a fourth-order Butterworth filter with a cut-off frequency such as 30 Hz and 60 Hz respectively.
